(19) United States
(12) Patent Application Publication
(10) Pub. No.: US 2005/0106132 A1
(43) Pub. Date: May 19, 2005

(54) Growth promoting prebiotic for lactobacillus dietary supplements

(76) Inventor: Randolph S. Porubcan, Victoria, MN (US)

Correspondence Address: Eric Mirabel;Bioarrays, 35 Technology Drive, Warren, NJ, 07059, US

(21) Appl. No.: 10/915,154

(22) Filed: Aug. 9, 2004

Related U.S. Application Data
(60) Provisional application No. 60/495,558, filed on Aug. 14, 2003.

(51) Int. Cl. A61K 45/00; C12N 1/20

(52) U.S. Cl. 424/93.45; 435/253.6

(57) ABSTRACT

Polyoxyethlene sorbitan monooleate (“PSM” or “polysorbate 80”) can be used in dietary supplements as a prebiotic for the stimulation of probiotic bacteria growth, even when present in only milligram quantities, e.g., 5-100 mg/dose. However, being a viscous liquid, PSM must be dried before incorporation. Moreover, it has been demonstrated that water activity in a probiotic formulation will greatly reduce the shelf-life of the formulation. Thus, even once the PSM is dried, extended shelf-life can be gained by reducing the water activity of the entire formulation. Disclosed herein is the use of dry PSM in formulations to enhance growth of probiotics, and reducing the formulation water activity to avoid reducing shelf-life.

### FIELD OF THE INVENTION

This Application claims priority to U.S. Provisional Application Ser. No. 60/495,558, filed Aug. 14, 2003.

### BACKGROUND

Dietary supplements that contain viable probiotic bacteria are increasing in popularity as the public becomes educated regarding their health benefits. These benefits are wide ranging and, in addition to supporting intestinal health and function, include repopulating the gut after antibiotic therapy, as well as offsetting lactose intolerance, supporting the immune system and reducing cholesterol. Lactic acid bacteria, primarily from the Lactobacillus and Bifidobacterium genera, that are capable of improving or maintaining intestinal health and function, are regarded as probiotic bacteria. For use in commercial dietary supplements, probiotic bacteria (also known as “probiotics”) can be grown commercially in stainless steel fermentors in various growth media, followed by harvesting and freeze-drying. Two of the most frequently used microbiological growth media are MRS broth and LBS broth; both contain glucose, peptones, yeast extract, various mineral salts, sodium acetate and potassium phosphate buffers, and polysorbate 80, which is an oily, viscous liquid. Together, these microbial nutrients effectively satisfy the fastidious nutritional requirements of probiotic bacteria.

When probiotics are ingested they must grow and multiply in the intestinal tract without the benefit of microbiological growth media. Therefore, probiotic growth in the intestinal tract depends to a large extent on the nutrients present in the patient's diet. Typical human diets are not well suited for probiotics and, given the abundance of and competition from many less fastidious intestinal bacteria, it can be difficult for probiotics to effectively multiply in vivo. To help correct this problem, manufacturers of probiotic dietary supplements have started to include prebiotics in their formulations.

Prebiotics are nutrient substances that encourage the growth of probiotics in vivo. Many are not digested or absorbed in the small intestine but pass into the colon where they stimulate the growth of probiotic bacteria, particularly Bifidobacteria. Fructo-oligosaccharides (FOS) are one type of prebiotic; inulin compounds (which are also oligosaccharides) are another. For these compounds to be effective they must be ingested in relatively large quantities, such as 4-10 grams/day for FOS and 10-14 grams/day for inulin. Probiotics, by comparison, can be effectively administered in milligram quantities containing 10⁷-10¹⁰ colony forming units (cfu). Thus, it is impractical to mix FOS or inulin with probiotics and deliver them in capsules or tablets.

The effects of carbohydrate type prebiotics may not always be beneficial, as they can encourage the growth of non-probiotic bacteria, as indicated in an article entitled “Culture-Independent Microbial Community Analysis Reveals that Inulin in the Diet Primarily Affects Previously Unknown Bacteria in the Mouse Cecum (Appl. Envir. Microbiol. 68: 4986-4995). FOS can cause flatulence and abdominal pain and some people experience severe allergic reactions to inulin. Therefore, there is a need for a non-carbohydrate prebiotic that can be used at low dosage while effectively stimulating probiotic bacteria.

### SUMMARY

Polyoxyethlene sorbitan monooleate (“PSM” or “polysorbate 80”) can be used in dietary supplements as a prebiotic for the stimulation of probiotic bacteria growth, even when present in only milligram quantities, e.g., 5-100 mg/dose. However, being a viscous liquid, PSM cannot be directly incorporated into dry probiotic formulations without causing substantial destruction of the probiotic bacteria. It must be dried before incorporation. Moreover, it has been demonstrated that water activity in a probiotic formulation will greatly reduce the shelf-life of the formulation. Thus, even once the PSM is dried, extended shelf-life can be gained by reducing the water content of the entire formulation. It is demonstrated herein that unless the PSM water activity is reduced to a substantial degree by other ingredients which absorb water, the shelf-life will be reduced, as measured by the bacterial colony forming units present after storage periods of from 30 days to 6 months. Therefore, it is preferable if the PSM be absorbed into a dry free-flowing powder with properties which aid in compatibility, prior to its inclusion in a probiotic blend. The dry free-flowing powder can be generated by adding an ingredient known as UOP, or by drying all the formulation ingredients, including the PSM.

### BRIEF DESCRIPTION OF THE DRAWINGS

FIG. 1 shows turbidity plots for three examples of probiotic bacteria grown in the presence and absence of PSM (Tween 80). PSM dramatically stimulates the growth of Lactobacillus paracasei (F-19) and Lactobacillus rhamnosus HN001 (HRU). Higher turbidity values (NTU) indicate greater cell density and growth.

### DETAILED DESCRIPTION

Probiotic Cultures

Six commercial, freeze-dried probiotic cultures were used to demonstrate the effectiveness of PSM as a prebiotic: 1) Lactobacillus paracasei strain F-19, Medipharm, Inc., Des Moines, Iowa; 2) Lactobacillus rhamnosus HOWARU strain HN001, Danisco A/S, Brabrand, Denmark; 3) Bifidobacterium lactis HOWARU strain HN019, Danisco A/S, Brabrand Denmark; 4) Bifidobacterium bifidum strain BB-12, Chr. Hansen A/S, Horsholm, Denmark; 5) Lactobacillus acidophilus strain LA-1; Chr. Hansen, Inc., Milwaukee, Wis.; 6) Lactobacillus casei strain 163; Danisco, Milwaukee, Wis.

Experiment 1

Demonstration of Probiotic Growth Stimulation by Polyoxyethlene Sorbitan Monooleate (PSM)

The six aforementioned probiotic cultures were inoculated, separately, into duplicate 90 ml aliquots of Special Activity Medium (ingredients listed below Table I) at 10⁷ cfU/ml. One set of aliquots was supplemented with 0.1% polyoxyethlene sorbitan monooleate (PSM), TWEEN 80K from EDC Industries, Inc. Elk Grove, Ill. All aliquots were then incubated at 37° C. and turbidity measurements, reported in NTU, were determined at the start and at intervals of 18, 24 and 46.5 hours using a Hach Turbidimeter model 2100N. The results are reported in Table 1 below.

TABLE 1

| | NTU During Incubation | | | |
|---|---|---|---|---|
| Culture @ 10⁷ cfu/ml @ T = 0 | 0 | 18 hr | 24 hr | 46.5 hr |
| Lactobacillus paracasei strain F-19 | 0 | −7 | 12 | 670 |
| Lactobacillus paracasei strain F-19 + | 0 | 136 | 739 | 2284 |
| PSM | | | | |
| Lactobacillus rhamnosus strain HN001 | 0 | 40 | 178 | 919 |
| Lactobacillus rhamnosus strain | 0 | 53 | 749 | 1592 |
| HN001 + PSM | | | | |
| Bifidobacterium lactis strain HN019 | 0 | 10 | 27 | 812 |
| Bifidobacterium lactis strain HN019 + | 0 | −2.5 | 0 | 8.5 |
| PSM | | | | |
| Bifidobacterium bifidum strain BB-12 | 0 | 7.5 | 14 | 621 |
| Bifidobacterium bifidum strain BB-12 + | 0 | −5.75 | −2.55 | −3.3 |
| PSM | | | | |
| Lactobacillus acidophilus strain LA-1 | 0 | −10 | −6 | 220 |
| Lactobacillus acidophilus strain LA-1 + | 0 | 38 | 963 | 2688 |
| PSM | | | | |
| Lactobacillus casei strain 163 | 0 | −4 | 20 | 770 |
| Lactobacillus casei strain 163 + PSM | 0 | 144 | 816 | 2424 |

Special Activity Medium Ingredients and Preparation

| | | |
|---|---|---|
| Tastone 154 (Sensient, Inc.) | 2.5 | g |
| Amber EHC (Sensient, Inc.) | 2.5 | g |
| Glucose (anhydrous) | 5.0 | g |
| Disodium phosphate | 0.5 | g |
| Tap water | 1,000 | ml |

Autoclave at 121° C. for 30 min. at pH 6.5.

This experiment shows that PSM stimulates the growth of Lactobacillus probiotic bacteria, measured in NTU. The higher the NTU number the more turbid the sample and the more bacterial growth. The two strains of Bifidobacterium, however, were not stimulated by PSM.

Experiment 2

Effect of Direct Mixing of PSM with Dry Probiotics on Water Activity (aw) and Viable Plate Count

Liquid PSM was mixed with freeze dried probiotic powders at 5% by weight (25 mg/500 mg) and water activity and plate counts (MRS agar incubated with the probiotics at 37° C., 3 days in H₂/CO₂ atmosphere) were determined at T=0 and T=1 month. Colony Forming Units (CFU) are reported at 10⁹/g.

The following freeze dried Lactobacillus cultures were used for this experiment: Lactobacillus acidophilus LA-1, 207×10⁹/g, aw=0.02; Lactobacillus paracasei F-19, 200×10⁹/g, aw=0.02; Lactobacillus rhamnosus HN001, 100×10⁹/g, aw=0.02.

TABLE II
(CFU reported at 10⁹/g)

| | | CFU @ | aw @ | CFU @ |
|---|---|---|---|---|
| Sample | aw @ T = 0 | T = 0 | T = 1 mo. | T = 1 mo. |
| LA-1 + 5% PSM | 0.07 | 196 | 0.08 | 1.2 |
| F-19 + 5% PSM | 0.08 | 188 | 0.08 | 0.92 |
| HN001 + 5% PSM | 0.07 | 93 | 0.07 | 0.77 |

It can be seen that the direct addition of PSM to probiotic powders has a significant negative effect on the plate counts after storage at 25° C. for one month.

Experiment 3

Sensitivity to Water Activity for Dry Probiotics Stored at 25° C.

The freeze-dried probiotic cultures described in Experiment 1 were mixed as follows: A. (A-BLENDS) 10 grams culture powder+70 grams microcrystalline cellulose (Avicel PH112, FMC Corp.)+20 grams sodium alginate (Colloid 488T, Tic Gums, Inc.) as received from manufacturer (14% moisture); B (B-BLENDS) Same as A-BLENDS except the sodium alginate was dried in a vacuum oven at 50° C. for 8 hours to 3.2% moisture. The average water activity was 0.12 for the A-Blends and 0.045 for the B-Blends. The mixed cultures were stored in amber glass bottles at 25° C. for 90 days; plate counts (reported in cfu/gram) were made on MRS agar as in Experiment 1 at T=0 and at T=90 days. The results are reported below in TABLE III.

TABLE III

| | A-Blends | B-Blends |
|---|---|---|
| | aw = 0.12 | aw = 0.045 |
| | T = 0 cfu/T = 90 cfu | T = 0 cfu/T = 90 cfu |
| Lactobacillus paracasei | 20 × 10⁹/10 × 10⁷ | 20 × 10⁹/16 × 10⁹ |
| strain F-19 | | |
| Lactobacillus rhamnosus | 10 × 10⁹/5 × 10⁶ | 10 × 10⁹/9 × 10⁹ |
| strain HN001 | | |
| Bifidobacterium lactis | 57 × 10⁹/3 × 10⁷ | 57 × 10⁹/33 × 10⁹ |
| strain HN019 | | |
| Bifidobacterium bifidum | 82 × 10⁹/2 × 10⁸ | 82 × 10⁹/78 × 10⁹ |
| strain Bb-12 | | |
| Lactobacillus acidophilus | 42 × 10⁹/4 × 10⁵ | 42 × 10⁹/32 × 10⁹ |
| strain La-1 | | |
| Lactobacillus casei | 160 × 10⁹/24 × 10⁹ | 160 × 10⁹/155 × 10⁹ |
| strain 163 | | |

It can be seen that with the possible exception of L. casei 163, a water activity of 0.12 in the mixture substantially reduces probiotic shelf-life at 25° C. However, when water activity is 0.045, the mixture has an acceptable shelf-life.

Although it is desirable to produce dry probiotic formulations that contain PSM in quantities sufficient to stimulate the in vivo growth of said probiotic bacteria, it is clear that because of the negative effects of PSM addition on shelf-life, the production process of the formulation needs to be controlled, so that the addition of PSM does not de-stabilize the shelf-life of the resulting formulations.

In order to reduce the water activity and avoid unacceptable reductions in product shelf-life, a requirement for the production process is a low humidity room with relative humidity controlled at 20% (+/−5%). Similarly, a vacuum drier capable of drying powders in trays at low temperatures (40-70° C.) at vacuums ranging from 24-29 inches of Hg is also required. Suitable vacuum driers include the LabLine Model 3620 available from Lab-Line Instruments, Inc., Melrose Park, Ill. An instrument for measuring water activity in powders is generally also required, and an acceptable unit is the Rotronic Hygromer Model A2 available form Rotronic Instrument Corp., Huntington, N.Y.

In general, to insure that an acceptable water activity (e.g., in the range of 0.01 to 0.05) is achieved in the final blend, the PSM ingredient should be uniquely prepared as a dry free flowing powder with a low water activity prior to mixing with dry probiotic bacteria. When PSM is admixed with dry probiotic bacteria, such as freeze-dried bacteria, in the form of a viscous liquid (as it is normally obtained from suppliers) the resulting mixtures have high water activities (>0.10) that significantly destabilize the probiotic bacteria during storage either at room temperatures (65-75° F.) or at refrigeration temperatures (35-45° F.). Such mixtures are not suitable for use as commercial probiotic powders for tableting or encapsulation.

There are various additives for rendering the liquid PSM into an acceptable dry, free flowing powder form such that, when the PSM is admixed with dry probiotic bacteria, it does not destabilize the bacteria. These ingredients or combination of ingredients result in powders having low water activities e.g., below 0.05. Examples of ingredients that can be used directly from the manufacturer and produce acceptable final blends with acceptable shelf-lives include compounds that are very effective adsorbents of water or water vapor, e.g., UOP T Powder and UOP L Powder (A.B. Colby, Inc., McMurray, Pa.) and Sylosiv 120 or Sylosiv A4 (W.R. Grace & Co., Columbia, Md.). All four of these substances are synthetic, molecular sieve, zeolites comprised of sodium, calcium or potassium aluminosilicates. Examples of other additive ingredients, also for absorbing water, that generally must be treated to remove water prior to use in order to produce acceptable shelf-lives in the final blend are various food starches, silicon dioxide, calcium silicate, clays such as kaolin and sodium bentonite, hydrocolloid gums such as sodium alginate, guar gum, gum Arabic and carrageenin and certain protein substances such as sodium caseinate. If the resulting probiotic product is destined for human use, then the ingredients used to render the PSM must be food grade substances. If the probiotic is destined for animal use the ingredients must conform to animal feed ingredient standards such as those approved by the American Association of Feed Control Officials (A.A.F.C.O.).

Treatment of the forgoing additive ingredients to make them suitable for blending with PSM so that a dry, low water activity, powder is produced involves activating them with heat so as to increase their ability to absorb water or water vapor, and to increase their affinity for water beyond that of the probiotic powders that will ultimately be added to the final mixture. In this way the water that is part of the PSM will stay within the additive ingredient and will not migrate back to the probiotics resulting in destabilization. The best way to treat or activate such ingredients is by heating them. Ingredients that are heat sensitive, such as hydrocolloid gums, are preferably activated in a vacuum oven, which can be operated at relatively low temperatures while drawing a vacuum on the product being heated. For purposes of the present invention it is acceptable to heat sensitive ingredients at 50-70° C. for 12-24 hours or longer in a vacuum oven operating at 24-29 inches of mercury. Food starches, such as corn starch and potato starch, treated in this manner have residual moisture contents of 2-5% and are quite effective in a final blend for use in rendering PSM into a dry, free flowing, powder with low water activity (below Aw=0.05). Optionally, low temperature infrared convection drying, where the maximum temperature that does not result in heat denaturation of the ingredient, can be used for heat sensitive ingredients. Ingredients that are not heat sensitive such as silicon dioxide, calcium silicate and various clay substances can be activated in a conventional oven at high temperatures such as 350-450° F. for 12-18 hours. The additive ingredients are not limited to those listed above, but include any human food grade or animal feed grade substance that is capable of producing a dry free flowing powder of low water activity (below 0.1) when mixed with PSM.

All steps of final blend preparation, including handling and blending, should be done in a low humidity room with the humidity controlled at 20% (+/−5%). Typically, the viscous PSM is poured into the dry additive ingredient (which is either heat activated or used as is, depending on which ingredient) while it is being mixed in a Hobart type, double action, rotary mixer. Mixing is conducted for as long as necessary to create a homogeneous mixture; usually 30-60 minutes is required. Since the PSM is viscous and sticky, it may be necessary to use a rubber spatula to assist in scraping the sides of the mixing bowl and to keep the PSM moving while it is being absorbed by the rendering ingredient. The amount of PSM as a weight percent of the blend depends on the type of additive ingredient. For example, with heat activated potato starch, from 1-15% PSM can be incorporated into the starch with about 10% being optimal. With calcium silicate, such as Hubersorb 600 (J.M. Huber Corp., Havre de Grace, MD), as much as 70% PSM can be incorporated. Using UOP T Powder as the additive ingredient, 25% PSM can readily be incorporated. In some situations it may be desirable to use a combination of ingredients; e.g., an ingredient with maximum PSM adsorption capability (e.g, Hubersorb 600) and an ingredient with maximum water adsorption capability (e.g., UOP T Powder). For example, PSM could first be blended at 70% by weight with Hubersorb 600 and the resulting mixture further blended with UOP T Powder at 50% by weight resulting in a final mixture that contains 35% PSM. When combinations of ingredients are used it may not be necessary to heat activate an ingredient that may otherwise require activation if it were used alone. This is the case with Hubersorb 600 and UOP T Powder. See Example 1.

When the rendered PSM is blended with dry probiotic powders, such as freeze-dried powders, the resulting blends should have a water activity in the range of 0.01 to 0.05 for acceptable shelf life. A water activity below 0.01 is also acceptable but difficult to achieve in practice. Accurate water activity measurements can be made using instruments such as the Rotronic Hygromer Model A2 water activity measuring instrument. This instrument, or an equivalent model, is available from the Rotronic Instrument Corp., Huntington, N.Y.

It is preferred that all ingredients, including the probiotic culture powder(s), test within the water activity range of 0.01-0.05. In preparation, ingredients are weighed to their required weights in a humidity controlled room held at 20% relative humidity and blended under similar conditions in a Patterson-Kelly (P.K.) type twin cone blender (a porcelain mortar and pestle can be used for lab scale batches as long as care is taken not to damage the mixture). P.K. blenders impart minimum sheer to powders which is desirable. After blending, the product is hermetically sealed in steel drums until it can be encapsulated into gelatin or cellulose capsules (also carried out at low humidity).

The quantity of PSM per capsule in a human probiotic product will usually range from 0.2-50 mg depending on the degree of probiotic stimulation required. For a 500 mg net weight capsule this represents from 0.04% to 10% of the contents. The amount of PSM in the blend should be related to the number of viable probiotic colony forming units (CFU)/capsule, such that capsules with greater CFUs include a proportionally greater amount of PSM. An acceptable range per capsule is 0.2 mg to 2 mg PSM/billion CFU. Each Lactobacillus strain may have different requirements for PSM. Therefore, an empirical lab test may be needed to determine the optimum amount required in a product formulation.

Some exemplary formulations are set out in the examples that follow.

### EXAMPLE 1

Preparing Dry PSM Powders

PSM in the form of Tween-80 from ICI Americas Inc. (Uniqema division, New Castle, Del.) was rendered into dry, free flowing, powders by various methods, as indicated below. All PSM mixtures were made on a weight/weight basis in a 4 liter Hobart mixer at 100 rpm for 30 minutes in a humidity controlled room (20% RH). All mixtures were stored in hermetically sealed amber glass jars until used for further testing. All mixtures were free flowing, white to slightly off white powders except for the sodium bentonite mixtures, which were gray. Blending was carried out in a dry room (relative humidity=20%) in a dry mortar and pestle made of porcelain.

A) UOP L powder, as received from the manufacturer, was blended with 25% PSM. aw=0.01.

B) Hubersorb 600, as received form the manufacturer, was blended with 70% PSM. aw=0.46.

C) Hubersorb 600 was heat activated under an infrared lamp at 155° F. for 5 hours, cooled to 70° F. then blended with 70% PSM. aw=0.108.

D) Sample from B) was blended 50/50 with UOP L powder. aw=0.05.

E) Sample from C) was blended 50/50 with UOP L Powder. aw=0.025.

F) Potato starch (Perfectamyl D6—Avebe), as received from the manufacturer, was blended with 10% PSM. aw=0.15.

G) Potato starch (Perfectamyl D6—Avebe) was heated under an infrared lamp for 7 hours at 220° F. (14% moisture removed) and then blended with 10% PSM. aw=0.03.

H) Corn starch (Pure Dent B830, Grain Processing Corp., Muscatine, Iowa) was blended with 10% PSM. aw=0.25.

I) Corn starch (Pure Dent B830), was heated in a vacuum drier at 160° F. for 20 hours at 28” vacuum, then blended with 10% PSM. aw=0.05.

J) Sodium alginate (Keltone HV, ISP Technologies, Inc., San Diego, Calif.) was blended with 10% PSM. aw=0.55.

K) Sodium alginate (Keltone HV) was heated under an infrared lamp for 10 hrs at 220 F (8% moisture removed) then blended with 10% PSM. aw=0.08.

L) Gum Arabic (Sigma Chemical Co., St. Louis, Mo.), as received from the manufacturer, was blended with 10% PSM. aw=0.38.

M) Gum Arabic (Sigma) was heated in a vacuum drier at 160° F. for 20 hours at 28″ of vacuum, then blended with 10% PSM. aw=0.08.

N) Kaolin clay (Vanclay, R.T Vanderbilt Co., Norwalk, Conn.), as received from the manufacturer, was blended with 10% PSM. Aw=0.49.

O) Kaolin clay (Vanclay) was heated in an oven at 450° F. for 16 hours then cooled to 70° F. and blended with 10% PSM. Aw=0.12.

P) Sodium bentonite (Volclay, American Colloid Co., Arlington Heights, EL), as received from the manufacturer, was blended with 10% PSM. Aw=0.55.

Q) Sodium bentonite (Volclay) was heated in an oven at 450° F. for 16 hours, cooled to 70° F. then blended with 10% PSM. Aw=0.09.

R) Silicon dioxide (Syloid 244 FP, W.R. Grace & Co., Columbia, Md.), as received from the manufacturer, was blended with 50% PSM. Aw=0.40.

S) Silicon dioxide (Syloid 244 FP) was heated in an oven at 450° F. for 16 hours, cooled, then blended with 50% PSM. Aw=0.35.

T) Sample from R) was blended 50/50 with UOP L Powder. Aw=0.04.

The only ingredient able to render PSM into a dry powder with a water activity of about 0.01 by direct blending was the UOP L powder. All other ingredients yielded significantly higher water activities when direct blended with PSM. Although their water activities could, at least in some cases, be reduced by adding UOP L powder (see formulations D and E above). Heat treatment, under an infrared lamp or in a vacuum drier, improved the ability of the various ingredients to yield acceptably low water activities when blended with PSM.

### EXAMPLE 2

Probiotic Stability

Freeze-dried powders of Lactobacillus acidophilus LA-1, Lactobacillus paracasei F-19 and Lactobacillus rhamnosus HN001 were blended 50% by weight with mixtures A-T from Example 1 and held in closed amber glass bottles for 6 months at 75° F. All samples were plated on MRS agar (plates incubated 72 hours at 37° C. in H₂/CO₂ atmosphere) and viable counts were reported in colony forming units (CFU)/gram at the start of the test (T=0) and after 6 mo. (T=6 mo.). All counts are reported as billion, (000,000,000), CFU/gm. The plate counts on the respective freeze-dried concentrates were: LA-1, 207×10⁹/gm; F-19, 200×10⁹/gm; HN001, 100×10⁹/gm.

TABLE IV

| | | CFU/gm @ | CFU/gm @ |
|---|---|---|---|
| | | T = 0 | T = 6 mo. |
| PSM Mixture | Lactobacillus Culture | (× 10⁹) | (× 10⁹) |
| A | LA-1 | 103 | 94 |
| A | F-19 | 100 | 96 |
| A | HN001 | 52 | 47 |
| B | LA-1 | 101 | 11 |
| B | F-19 | 98 | 7.2 |
| B | HN001 | 48 | 6 |
| C | LA-1 | 101 | 22 |
| C | F-19 | 99 | 15 |
| C | HN001 | 50 | 13 |
| D | LA-1 | 104 | 90 |
| D | F-19 | 104 | 88 |
| D | HN001 | 51 | 45 |
| E | LA-1 | 103 | 94 |
| E | F-19 | 100 | 90 |
| E | HN001 | 50 | 49 |
| F | LA-1 | 101 | 20 |
| F | F-19 | 102 | 17 |
| F | HN001 | 52 | 10 |
| G | LA-1 | 102 | 91 |
| G | F-19 | 98 | 90 |
| G | HN001 | 49 | 50 |
| H | LA-1 | 102 | 16 |
| H | F-19 | 99 | 12 |
| H | HN001 | 51 | 9 |
| I | LA-1 | 99 | 91 |
| I | F-19 | 98 | 89 |
| I | HN001 | 49 | 47 |
| J | LA-1 | 102 | 9 |
| J | F-19 | 99 | 5 |
| J | HN001 | 52 | 4 |
| K | LA-1 | 102 | 27 |
| K | F-19 | 101 | 17 |
| K | HN001 | 51 | 16 |
| L | LA-1 | 103 | 12 |
| L | F-19 | 100 | 8 |
| L | HN001 | 51 | 7 |
| M | LA-1 | 100 | 24 |
| M | F-19 | 99 | 15 |
| M | HN001 | 49 | 14 |
| N | LA-1 | 104 | 10 |
| N | F-19 | 101 | 6 |
| N | HN001 | 52 | 7 |
| O | LA-1 | 104 | 24 |
| O | F-19 | 102 | 15 |
| O | HN001 | 51 | 14 |
| P | LA-1 | 101 | 8 |
| P | F-19 | 99 | 4 |
| P | HN001 | 49 | 5 |
| Q | LA-1 | 104 | 31 |
| Q | F-19 | 103 | 22 |
| Q | HN001 | 54 | 17 |
| R | LA-1 | 101 | 11 |
| R | F-19 | 100 | 8 |
| R | HN001 | 49 | 8 |
| S | LA-1 | 103 | 13 |
| S | F-19 | 101 | 9 |
| S | HN001 | 52 | 9 |
| T | LA-1 | 101 | 90 |
| T | F-19 | 101 | 85 |
| T | HN001 | 48 | 41 |

PSM mixtures (from Table 1) designated A, D, E, G, I, and T resulted in acceptable probiotic stabilities after 6 months. All others mixtures yielded unacceptable results after 6 months (CFU/gm was too low to be commercially viable).

### EXAMPLE 3

Commercial Probiotic Formulations

Examples of commercial formulations were made using selected PSM mixtures from Example 1. Freeze-dried probiotic culture comprised the remainder of the formulations.

The probiotic cultures as listed in Example 2 were used here, and each had a water activity of 0.02. Each formulation was blended to contain 25 mg of PSM per 500 mg of finished formulation. Blending was accomplished in a lab scale P.K. blender in a low humidity room (20% R.H.) at 50 rpm for 20 minutes. Bacterial plate counts (MRS agar) were in the range of 10¹⁰ to 10¹¹ per gram for the finished formulations.

Finished blends were filled into size “o” Vegicaps (Capsugel, Inc.) at a net weight of 445 mg per capsule and stored in sealed amber glass bottles at 25° C. for 6 months. The contents of sample capsules were tested for activity in Special Activity Medium after 6 months storage as indicated below.

Special Activity Medium—Activity Test

The special activity medium in Experiment 1 was prepared in 100 ml aliquots in screw cap erlenmeyer flasks and sterilized at 121° C. for 30 min. The flasks were inoculated with the PSM+probiotic formulations at 0.1% by weight and incubated at 37° C. Plate counts were made on MRS agar at T=0, 18, 24 and 48 hours.

Example Blends

1) PSM from A-blend (Ex. 1)+LA-1
2) PSM from B-blend+LA-1
3) PSM from F-blend+LA-1
4) PSM from G-blend+LA-1
5) PSM from A-blend (Ex. 1)+F-19
6) PSM from B-blend+F-19
7) PSM from F-blend+F-19
8) PSM from G-blend+F-19
9) PSM from A-blend (Ex. 1)+HN001
10) PSM from B-blend+HN001
11) PSM from F-blend+HN001

12) PSM from G-blend+HN001

TABLE V

| | | | | |
|---|---|---|---|---|
| CFU/ml @ | T = 0 | T = 18 hr | T = 24 hr | T = 48 hr |

Example blend 1) - Activity after 6 mo. @ 25° C.

| | | | |
|---|---|---|---|
| 1.7 × 10⁷ | 6.1 × 10⁷ | 3.2 × 10⁸ | 9.2 × 10⁸ |

Example blend 2) - Activity after 6 mo. @ 25° C.

| | | | |
|---|---|---|---|
| 9.0 × 10⁴ | 2.1 × 10⁵ | 3.0 × 10⁶ | 7.0 × 10⁶ |

Example blend 3) - Activity after 6 mo. @ 25° C.

| | | | |
|---|---|---|---|
| 7.1 × 10⁴ | 1.0 × 10⁵ | 1.9 × 10⁶ | 4.1 × 10⁶ |

Example blend 4) - Activity after 6 mo. @ 25° C.

| | | | |
|---|---|---|---|
| 1.3 × 10⁷ | 4.3 × 10⁷ | 2.2 × 10⁸ | 7.2 × 10⁸ |

Example blend 5) - Activity after 6 mo. @ 25° C.

| | | | |
|---|---|---|---|
| 1.9 × 10⁷ | 7.1 × 10⁷ | 4.2 × 10⁸ | 1.2 × 10⁹ |

Example blend 6) - Activity after 6 mo. @ 25° C.

| | | | |
|---|---|---|---|
| 2.0 × 10⁴ | 1.3 × 10⁵ | 1.9 × 10⁶ | 7.8 × 10⁶ |

Example blend 7) - Activity after 6 mo. @ 25° C.

| | | | |
|---|---|---|---|
| 1.2 × 10⁴ | 9.1 × 10⁴ | 2.3 × 10⁶ | 9.1 × 10⁶ |

Example blend 8) - Activity after 6 mo. @ 25° C.

| | | | |
|---|---|---|---|
| 1.8 × 10⁷ | 6.2 × 10⁷ | 3.9 × 10⁸ | 1.1 × 10⁹ |

Example blend 9) - Activity after 6 mo. @ 25° C.

| | | | |
|---|---|---|---|
| 8.2 × 10⁶ | 2.0 × 10⁷ | 3.8 × 10⁸ | 8.4 × 10⁸ |

Example blend 10) - Activity after 6 mo. @ 25° C.

| | | | |
|---|---|---|---|
| 1.4 × 10⁴ | 9.2 × 10⁴ | 8.7 × 10⁵ | 1.2 × 10⁶ |

Example blend 11) - Activity after 6 mo. @ 25° C.

| | | | |
|---|---|---|---|
| 1.1 × 10⁴ | 5.6 × 10⁴ | 6.1 × 10⁵ | 9.6 × 10⁵ |

Example blend 12) - Activity after 6 mo. @ 25° C.

| | | | |
|---|---|---|---|
| 7.8 × 10⁶ | 2.1 × 10⁷ | 4.2 × 10⁸ | 1.1 × 10⁹ |

PSM from the A and G blends of Example 1, when mixed with Lactobacillus probiotics, produced acceptable microbial activity after 6 months storage at 25° C.

The invention includes many variations, modifications and alterations of the embodiments and methods described in the above specification, and the scope of invention is not defined or limited by this specification or by the examples, but is defined only in the claims that follow, and includes all equivalents of the subject matter of the claims.

1. A formulation for stimulating the growth and reproduction of probiotic bacteria which avoids substantial destabilization of the bacteria during storage comprising, in addition to probiotic bacteria, dry polyoxyethylene sorbitan monooleate (“PSM”).

2. The formulation of claim 1 wherein the formulation is prepared so that the water activity of the final formulation does not exceed 0.05.

3. The formulation of claim 1 wherein the water activity of the final formulation is from 0.01 to 0.05.

4. The formulation of claim 1 wherein the PSM is added in an amount to make 1 to 40% of the weight of the final formulation and wherein said bacterial powder contributes from one million to one trillion colony forming units (CFU) per gram of final formulation.

5. A formulation of claim 1 where the PSM is rendered, by blending with dry powdered substances, into a dry powder with a water activity in the range of 0.01 to 0.05 prior to blending it with said probiotic bacteria.

6. A formulation of claim 5 wherein the dry powdered substances are one or more of silicon dioxide, calcium silicate, and aluminosilicate zeolytes, including sodium, potassium and calcium aluminosilicates.

7. A formulation of claim 5 where the dry powdered substances are starches or pectins.

8. A formulation of claim 5 where the powdered substances are naturally occurring clays or clay substances.

9. A formulation of claim 5 where the powdered substances are hydrocolloids or gums.

10. A formulation according to claims 5-9 where said powdered substances are dried to lower their water activity prior to blending with said probiotic bacteria.

11. A formulation according to claim 10 where the method of drying is by infrared convection drying or vacuum drying.

12. A process according to claim 1 where the probiotic bacteria are powdered, before formulation, by freeze drying or lyophilization.

13. A process according to claim 1 where the probiotic bacteria are Lactobacillus species.

14. A process according to claim 1 where the probiotic bacteria are pre-blended with a dry powdered substance to standardize the CFU/gram prior to blending with PSM.

15. A blend of dry PSM and probiotics.

16. The blend of claim 15 wherein the water activity of the blend is less than 0.05.

17. The blend of claim 16 wherein the prebiotics in the blend are not degraded or destroyed during storage to the extent that the blend is not commercially viable after 6 months.

18. The blend of claim 17 which retains more than 80% of the starting probiotics after 6 months

19. The blend of claim 16 where the blend includes UOP powder or is dried

20. The blend of claim 19 where the blend includes additives including one or more of silicon dioxide, calcium silicate, aluminosilicate zeolytes, including sodium, potassium and calcium aluminosilicates, starches or pectins, clays or clay substances or hydrocolloids or gums.

21. The method of claim 20 wherein the additives are dried prior to inclusion in the blend by infrared convection drying or vacuum drying.

22. The method of claim 20 wherein gums and starches are not among the additives and the additives are vacuum dried at a temperature of 350-450° F. for 12-18 hours.

23. The method of claim 22 wherein gums and starches are among the additives and the additives are dried at 50-70° C. for 12-24 hours or longer in a vacuum oven operating at 24-29 inches of mercury.

24. A method of avoiding reductions in the shelf life of probiotics blended with PSM by reducing the water activity of the blend to below 0.05, by either adding a water absorbing agent or drying the blend.

25. The method of claim 23 wherein the agent is an aluminosilicate zeolite.

26. The method of claim 25 wherein the agent is UOP.

27. A method of administering probiotics to a subject comprising mixing the probiotics, before administration, with prebiotics including at least PSM, where the prebiotics have their water activity reduced below 0.05.
